(19) United States
(12) Patent Application Publication
(10) Pub. No.: US 2021/0007903 A1
(43) Pub. Date: Jan. 14, 2021

(54) Analgesic Nose Plugs

(71) Applicant: Rhinoclear Nasal Care Solutions Inc., Calgary (CA)

(72) Inventors: Bradford Mechor, Calgary (CA); Merle Olson, Calgary (CA)

(73) Assignee: Rhinoclear Nasal Care Solutions Inc., Calgary (CA)

(21) Appl. No.: 17/031,487

(22) Filed: Sep. 24, 2020

Related U.S. Application Data
(63) Continuation-in-part of application No. 16/345,452, filed on Apr. 26, 2019.
(60) Provisional application No. 62/413,527, filed on Oct. 27, 2016.

(51) Int. Cl. A61F 13/20; A61F 13/26

(52) U.S. Cl. CPC A61F 13/2074; A61F 2013/15081; A61F 13/26; A61F 13/2005

(57) ABSTRACT

Disclosed is a deformable analgesic nose plug, comprising a deformable core material and an outer coating provided on said deformable core material, wherein said outer coating comprises one or more analgesic compounds. The nose plug has a tapered distal end for insertion into a nostril and a proximal end whereby the nose plug is inserted into the nostril. The deformable core material may comprise a multi-phasic core material wherein a first phase of the multi-phasic core material is a solid or a semi-solid structure at a temperature of 35° C. or less and a second phase of the multi-phasic core material is characterized by liquefaction at a temperature of 37° C. or greater. The deformable core material may also comprise a porous resiliently compressible substrate. The deformable porous resiliently compressible core material may be one of polymeric sponges, naturally occurring sponges, fluffy cellulosic materials, cotton, rolled gauze, and mixtures thereof.

### CROSS-REFERENCE TO RELATED APPLICATIONS

This application is a continuation-in-part application of U.S. patent application Ser. No. 16/345,452, entitled “Hemostatic Nose Plugs”, filed on Apr. 26, 2019, which is a 371 National Stage of International Application No. PCT/CA2017/051285, entitled “Hemostatic Nose Plugs”, filed on Oct. 27, 2017, which claims priority to and the benefit of U.S. Provisional Patent Application No. 62/413,527, entitled “Hemostatic Nose Plugs”, filed on Oct. 27, 2016, and the specification and claims thereof are incorporated herein by reference.

### TECHNICAL FIELD

This disclosure generally relates to non-invasive techniques for delivering medicaments. In particular, the disclosure relates to hemostatic-coated nose plugs configured to deliver medicaments through intranasal pathways.

### BACKGROUND

Intranasal pathways may be used to deliver certain medicaments to the bloodstream. In more detail, the nasal cavity is covered by a thin mucosa that is well-vascularized. Medicaments administered via the intranasal pathways thus need only transfer across a single epithelial cell layer for systematic blood circulation. As well, because the medicaments are administered via the intranasal pathways, they avoid the first pass effect, which simplifies the determination of their dosages. In some cases, the medicaments may also avoid the blood brain barrier.

Medicaments designed for intranasal delivery are typically incorporated into nasal sprays. The nasal sprays produce a mist of a solution containing the medicament that coats the nostril and/or nasal passage. The medicament is then transferred across the mucosa and into the bloodstream. However, as will be appreciated, nasal sprays are not well suited for prolonged delivery of a medicament, as the coating provided by the mist is quickly degraded by a user's breathing. As a result, several applications may be necessary. Further, it may therefore also be difficult to determine if an appropriate dose has been delivered.

One approach to addressing the limitations of medicament delivery via nasal spray has been to coat a cotton ball or the like in a medicament and insert the coated cotton ball into a patient's nose. The coated cotton ball contacts the inside of the nostril and/or nasal passage to deliver the medicament thereto. However, as will be appreciated, it is also difficult to determine the dosage administered to a patient, as it may be challenging to determine how much of the medicament is coated onto the cotton ball as well as the surface area of the cotton ball that is in contact with the patient's nostril and/or nasal cavity to deliver the medicament thereto.

### BRIEF SUMMARY

The present disclosure relates to analgesic nose plugs for treating and/or preventing pain and optionally other ailments.

According to one example embodiment of the present disclosure, there is provided a deformable analgesic nose plug. The deformable analgesic nose plug comprises a deformable core material comprising a multi-phasic core material. The multi-phasic core material has a first phase characterized by a solid or a semi-solid structure at a temperature of 35° C. or less and a second phase characterized by liquefaction at a temperature of 37° C. or greater. The deformable analgesic nose plug also comprises one or more analgesic compounds. In one aspect, the nose plug has a tapered distal end for insertion into a nostril and a proximal end whereby the nose plug is inserted into the nostril.

The analgesic nose plugs of the present disclosure may provide a number of advantages. For example, the nose plugs may provide rapid relief of pain such as head, face, and/or neck pain. In more detail, medicaments administered intranasally may be transferred directly to the blood stream via the mucosa. However, most over-the-counter treatments available are for administration in the form of pills or tablets. Such treatments may, in contrast, take up to an hour to provide relief of pain or other related symptoms, depending on when the patient last ate, how much they ate, and other such events. The nose plugs of the present disclosure are not affected by such factors and may consistently provide rapid administration of medicaments and thus rapid relief of pain.

Another advantage of the nose plugs of the present disclosure is that they may deliver medicaments non-invasively. As described above, treatments administered by way of pills or tablets may take a significant amount of time to take effect. One alternative used by medical practitioners is to administer the treatment intravenously by way of a needle, which affords a more rapid delivery of the medicament than the pills or tablets. However, needles are uncomfortable and may be painful. The nose plugs of the present disclosure, in contrast, may provide similar delivery of medicaments to the bloodstream while also avoiding the discomfort and pain caused by needles.

Yet another advantage of the nose plugs of the present disclosure is that they may provide prolonged administration of medicaments to a patient. In more detail, the nose plugs are inserted into a patient's nose and allowed to deliver medicaments for an extended period of time, which may be particularly useful for anesthetics. Conventionally, for prolonged administration, medicaments are administered intravenously, which has a number of disadvantages such as those described above.

Further advantages will be discussed below and will be readily apparent to those of ordinary skill in the art upon reading the present disclosure.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. The embodiments of the present disclosure will be described with reference to the following drawings in which:

FIG. 1A is a side view of the nose plug according to one embodiment of the present disclosure, and FIG. 1B is a cross-sectional view of the nose plug;

FIG. 2 is a cross-sectional side view of another embodiment of another nose plug according to of the present disclosure;

FIG. 3 is a cross-sectional side view of another embodiment of another nose plug according to the present disclosure; and

FIG. 4 is a cross-sectional side view of another embodiment of another nose plug according to the present disclosure.

### DETAILED DESCRIPTION

Some embodiments of the present disclosure generally pertain to nose plugs having hemostatic coatings thereon for affecting immediate cessation of nosebleeds upon insertion of the nose plugs into nasal cavities. According to one aspect, the present nose plugs comprise a core material for receiving and supporting thereon a coating comprising a compound with hemostatic properties. According to another aspect, each of the present nose plugs may additionally comprise an applicator stick engaged with the core material. Alternatively, each of the present nose plugs may comprise a pliable material engaged with the core material wherein a portion of the pliable material extends outward from the core material.

As used herein, “nose plug” refers to a device to be inserted into a nostril. The nose plugs generally sized such that, when inserted into the nostril, the nose plug contacts the inner surfaces of the nostril in order to maintain its position therein.

As used herein, the term “core material” refers to the material or materials that form the body of the nose plugs of the present disclosure. The core material is deformable in that it is capable of being reshaped, which facilitates the insertion of the nose plugs into nostrils of different sizes.

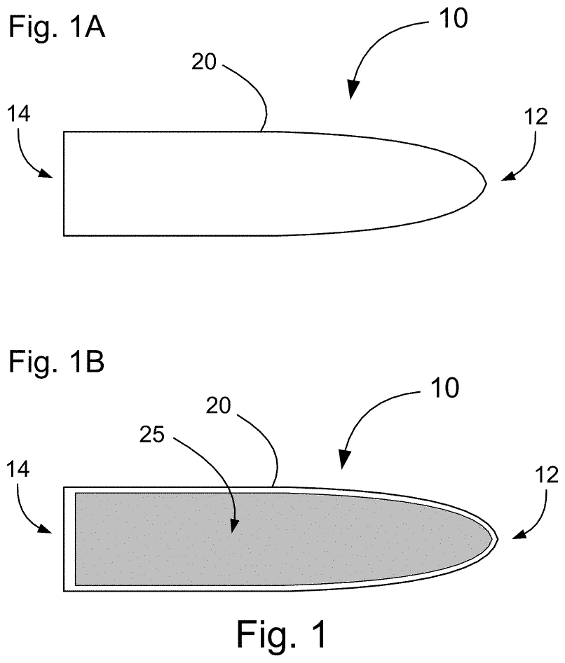

An embodiment of a nose plug 10 according to the present disclosure is shown in FIGS. 1A and 1B and comprises a multi-phasic core material 25 that has been coated with a hemostatic composition 20.

A suitable multi-phasic core material 25 may be formed from a mixture of materials that are solids or semi-solids at temperatures lower than about 36° C. (i.e., a first phase) and that liquefy at a temperature of 37° C. or higher (i.e., a second phase). Examples of suitable mixtures of materials for preparing the multi-phasic core material 25 include of two or more of cocoa butter, polyethylene glycols, hydrogels, glycerin, polyglycolysed glycerides, and glycerinated gelatin. Suitable polyglycolysed glycerides include but are not limited to arachidic acid, lauric acid, myristic acid, aleic acid, palmitic acid, and steric acid. The core material 25 may additionally comprise an excipient exemplified by monodecanoyl-glycerol, monolauroyl-glycerol, fatty acid triglycerides, thylene glycol, triethylene glycol, propylene glycol, butylene glycols, 1,2-propylene glycol, and the like. The multi-phasic core material 25 may additionally comprise a lubricant exemplified by mineral oil, vegetable oil, polyisobutene, polyalkyleglycol, polyolefin, diester, polymerester, vegetable stearin, magnesium stearate, hydrogenated vegetable oils, cocoa butter, theobroma oil, hydrogenated corn oil, palm oil, palm kernel oil, coconut oil, stearic acid, and the like. The multi-phasic core material 25 may additionally comprise an emollient exemplified by salicylic acid, ammonium lactate, urea, dimenticone, and the like.

The multi-phasic core material may be prepared by mixing together the selected materials in heated water, for example, at a temperature from a range of 40° C. to 90° C. and anywhere therebetween, and then pouring the mixture into molds for curing and forming. One example of a suitable mixture of material for the multi-phasic core is about 14% gelatin plus about 70% glycerol plus about 16% water. Another example of a suitable mixture is about 33% PEG 4000 plus about 47% PEG 6000 plus about 20% water. Suitable molds for forming the multi-phasic core material into nose plugs include metal molds, flexible rubber molds, silicone molds, and the like. Alternatively, the multi-phasic core material may be formed into the nose plug 10 using a suppository-manufacturing device, which are known in the art. Each nose plug of the present disclosure generally comprises about 0.5 g, about 0.6 g, about 0.7 g, about 0.8 g, about 0.9 g, about 1.0 g, about 1.1 g, about 1.2 g, about 1.3 g, about 1.4 g, about 1.5 g of material and therebetween. Particularly suitable is about 1.0 g of core material.

The nose plugs of the present disclosure generally have an elongate cylindrical shape with a tapered conical distal end 12 (i.e., the end that is inserted into a nostril). Such shapes are commonly referred to as bullet-shaped or torpedo-shaped. It is optional for the distal end 12 of the nose plugs 10 to have a frusticonical taper terminating with a rounded end. The proximal end 14 of a nose plug 10 of the present disclosure, may be flat. Alternatively, the proximal end of the nose plug may be concave. Alternatively, the proximal end of the nose plug may be convex. The diameter of the multi-phasic core material of the nose plugs 10 may be selected from a range of about 0.5 cm to about 1.75 cm and therebetween. The diameter of a nose plug 10 approximate its proximal end 14 may be same as the diameter of the central body portion of the nose plug 10, i.e., the nose plug 10 may have the same diameter from its proximal end 14 until the onset of tapering to its distal end 12. Alternatively, the diameter of a nose plug according to the present disclosure, may be slightly flared outward approximate its proximal end. Alternatively, the diameter of a nose plug according to the present disclosure, may be slightly flared outward approximate its proximal end. The length of the multi-phasic core material of the nose plugs may be selected from a range of about 1.5 cm to about 3.5 cm and therebetween. The multi-phasic nature of the core material makes it deformable as the present nose plug is inserted into a nostril so that a large portion of the outer covering of the nose plug is in at least partial and preferably full contact with the nasal membranes lining the nostril.

After the multi-phasic cores of the nose plugs have been formed and solidified, a solution comprising one or more hemostatic compounds is coated onto the outer surface of the nose plug cores, for example, by dipping or by spraying or other suitable coating processes, after which the coated nasal plugs are dried. Suitable hemostatic compounds include but are not limited to ferrous sulfate, ferric sulfate, aluminum sulfate, ammonium sulfate, aluminum potassium sulfate, aluminum chloride, tannic acid, zinc chloride, the like, and mixtures thereof. Particularly suitable hemostatic compounds are ferrous sulfate, ferric sulfate, and mixtures thereof.

For use to stop a nosebleed, the present nose plug may be taken out of its container and simply inserted into the nostril such that some or all of the elongate outer surface of the nose plug is in contact with the nasal tissues lining the nostril and/or nasal passage. The hemostatic compound in the outer coating of the nose plug will cause cessation of bleeding at a capillary source(s) within the nostril and/or nasal passage. Additionally, because the of the deformable nature of the multi-phasic core material and the resiliently compressible substrate, the present nose plugs will provide a compression pressure onto and about the bleeding areas within the nostril and nasal passage. After insertion of the present nose plug into a nostril, the core material will slowly liquefy as the nose plug is warmed to body temperature (i.e., 37° C.) and will slowly flow to the back of the nasal cavity, toward the pharynx from where it may be spit out, or alternatively, pass into the esophagus and moved to the stomach where it will digested. Once the nosebleed has stopped, the nose plug can be removed simply by blowing the nose.

It is within the scope of the present disclosure to incorporate components into the multi-phasic core material if so desired. Suitable components that may be incorporated including clotting factors such as thrombin, prothrombin, thromboplastin, fibrinogen, and the like; antibiotic compositions such as penicillin, cephalosporin, tetracycline, macrolides, and the like; antimicrobial agents such as ivermectin, thymol, benzoic acid, phenolic acid, sorbic acids, alcohols, benzethonium chloride, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, propylparaben, sodium propionate, thimerosalaminoclycosides, glycopepetides, macrocodes, quinolones, streptogramins, carbapenems, and the like; antihistamines such as chlorpheniramine, clemastine, brompheniramine, diphenhydramine, loratadine, cetirizine, fexofenadine, capsaicin, and the like; decongestants such as pseudoephedrine, phenylephrine, oxymetazoline, epinephrine, xylometazoline, cocaine, and the like; and combinations thereof.

Other suitable components for incorporation into the multi-phasic core material include emollients exemplified by mineral oil, mixtures of mineral oil and lanolin alcohols, cetyl alcohol, cetostearyl alcohol, petrolatum, petrolatum and lanolin alcohols, cetyl esters wax, cholesterol, glycerin, glyceryl monostearate, isopropyl myristate, isopropyl palmitate, lecithin, allyl caproate, althea officinalis extract, arachidyl alcohol, argobase EUC, butylene glycol, dicaprylate/dicaprate, acacia, allantoin, carrageenan, cetyl dimethicone, cyclome hicone, diethyl succinate, dihydroabietyl behenate, dioctyl adipate, ethyl laurate, ethyl palmitate, ethyl stearate, isoamyl laurate, octanoate, PEG-75, lanolin, sorbitan laurate, walnut oil, wheat germ oil, super refined almond, super refined sesame, super refined soyabean, octyl palmitate, caprylic/capric triglyceride, glyceryl cocoate, and the like. An emollient, if present, is present in the compositions described herein in an amount by weight of the composition of about 1% to about 30%, about 3% to about 25%, or about 5% to about 15%. Illustratively, one or more emollients are present in a total amount of about 1% by weight, about 2%, about 3%, about 4%, about 5%, about 6%, about 7%, about 8%, about 9%, about 10%, about 11%, about 12%, about 13%, about 14%, about 1%, about 16%, about 17%, about 18%, about 19%, about 20%, about 21%, about 22%, about 23%, about 24%, about 25%, about 26%, about 27%, about 28%, about 29%, or about 30%, and therebetween.

Other suitable components for incorporation into the multi-phasic core material include antioxidants exemplified by citric acid, butylated hydroxytoluene (BHT), ascorbic acid, glutathione, retinol, a-tocopherol, β-carotene, a-carotene, ubiquinone, butylated hydroxyanisole, ethyl enediaminetetraacetic acid, selenium, zinc, lignan, uric acid, lipoic acid, N-acetylcysteine, and the like. An antioxidant, if present, is present in the compositions described herein in a total amount selected from the range of about 0.025% to about 1.0% by weight.

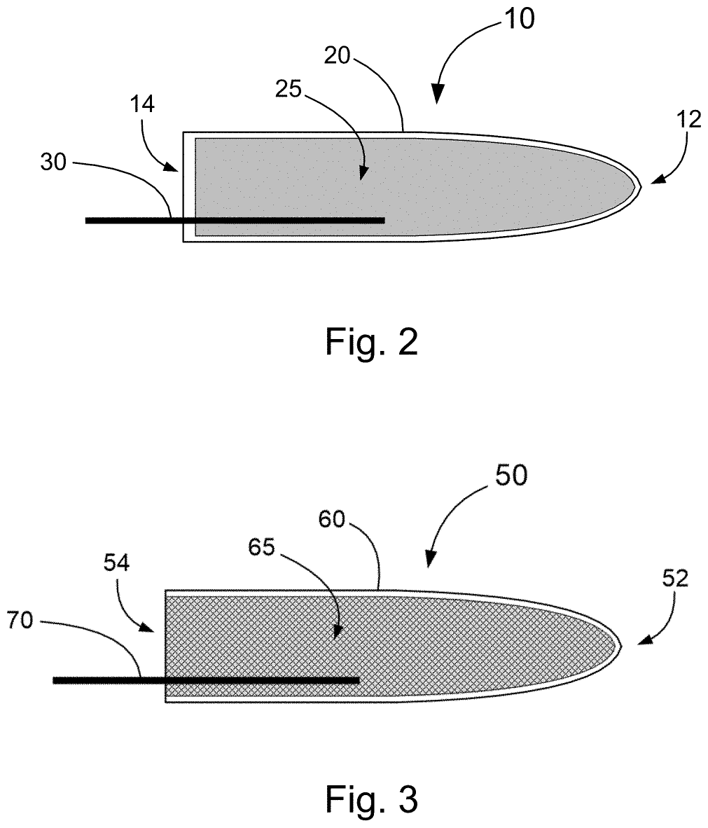

It is optional to insert an applicator stick 30 into the multi-phasic core material 25 of a nasal plug 10 as it is solidifying in a mold so that about 0.5 cm to about 1.5 cm of the applicator stick extends outward from the proximal end of the nasal plug (FIG. 2). The applicator stick 30 may be a wooden stick or a plastic stick or a polymeric stick, and the like. Alternatively, a length of a flexible material exemplified by a string, a cord, and the like, may be inserted into the multi-phasic core material of a nasal plug as it is solidifying in a mold so that a portion of about 0.5 cm to about 1.5 cm of the flexible material extends outward from the proximal end of the nasal plug. It is optional to place one end of a flexible material into one core material of a nasal plug as it is solidifying in a cavity of a mold and the other end of the flexible material into an adjacent core material in and adjacent cavity of the mold so that after the multi-phasic core material has solidified, two nose plugs are joined together by the flexible material that extends into both plug cores. Once the nosebleed has stopped, the nose plug can be removed via pulling on the applicator stick or the flexible material.

Another embodiment of a nose plug 50 according to the present disclosure is shown in FIG. 3. The nose plug 50 has a core material, a distal end 52, and a proximal end 54. The core material comprises a porous resiliently compressible substrate 65 into which has been imbedded an applicator stick 70 that extends outward from the proximal end 54 of the nose plug 50. The porous resiliently compressible core material 65 is coated with a selected hemostatic compound 60. Suitable hemostatic compounds include but are not limited to ferrous sulfate, ferric sulfate, aluminum sulfate, ammonium sulfate, aluminum potassium sulfate, aluminum chloride, tannic acid, zinc chloride, the like, and mixtures thereof. Particularly suitable hemostatic compounds are ferrous sulfate, ferric sulfate, and mixtures thereof.

Suitable porous resiliently compressible substrates include materials that can be formed to bullet shapes or torpedo shapes or elliptical shapes or cylindrical shapes, for example man-made polymeric sponges, naturally occurring sponges, fluffy cellulosic materials, cotton, rolled gauze, the like, and mixtures thereof. The porous core substrate 65 is coated with a hemostatic composition 60. Suitable hemostatic compositions include but are not limited to ferrous sulfate, ferric sulfate, aluminum sulfate, ammonium sulfate, aluminum potassium sulfate, aluminum chloride, tannic acid, zinc chloride, the like, and mixtures thereof. Particularly suitable hemostatic compounds are ferrous sulfate, ferric sulfate, and mixtures thereof. It is optional if so desired, to infiltrate the porous resiliently compressible substrate 65 with a multi-phasic core material prepared as previously described in reference to FIGS. 1A and 1B.

If so desired, the applicator stick 70 may be substituted for with a length of a flexible material exemplified by a string, a cord, and the like. It is optional to place one end of a flexible material into one porous resiliently compressible core material 65 of a nasal plug 50 and the other end of the flexible material into an adjacent porous resiliently compressible core material 65 so that after the porous resiliently compressible core materials 65 have been coated with a hemostatic composition 60, two nose plugs 50 are joined together by the flexible material that extends into both plug cores.

The resiliently compressible nature of the porous substrate 65 makes it deformable as the present nose plug is inserted into a nostril so that a large portion of the outer covering of the nose plug 50 is in full contact with the nasal membranes lining the nostril. Once the nosebleed has stopped, the nose plug 50 can be removed via pulling on the applicator stick 70 or the flexible material.

A further embodiment of the present disclosure relates to nose plugs comprising one or more compounds for treating and/or preventing pain and optionally other ailments. One example embodiment relates to a deformable analgesic nose plug, comprising: a deformable core material comprising a multi-phasic core material wherein a first phase of the multi-phasic core material is characterized by a solid or a semi-solid structure at a temperature of 35° C. or less, and wherein a second phase of the multi-phasic core material is characterized by liquefaction at a temperature of 37° C. or greater; and one or more analgesic compounds, wherein the nose plug has a tapered distal end for insertion into a nostril and a proximal end whereby the nose plug is inserted into the nostril.

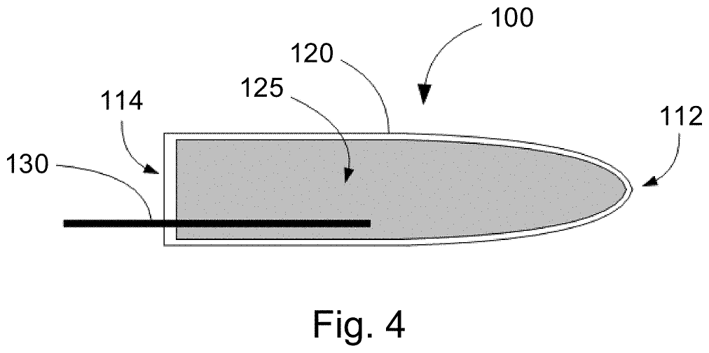

Referring to FIG. 4, there is shown an analgesic nose plug 100 according one embodiment of the present disclosure. The analgesic nose plug 100 comprises a deformable core material 125 and an optional outer coating of an analgesic composition 120. As well, the analgesic nose plug 100 has tapered distal end 112 and a proximal end 114. There is also illustrated an optional applicator 130 embedded into the nose plug 100. The nose plug 100, the tapered distal end 112 thereof, the proximal end 114 thereof, and the optional applicator 130 may be configured in the same manner as previously described herein in relation to the nose plugs 10, 50, the tapered distal ends 12, 52, the proximal ends 14, 54, and the applicators 30, 70, respectively.

The core material 125 of the analgesic nose plug 100 may also be configured in the same manner as the core materials 25, 65. For example, the core material 125 may comprise the previously described multi-phasic core material and/or the porous resiliently compressible core material.

In the illustrated embodiment, the core material 125 is coated with the analgesic composition 120. However, in other embodiments, the analgesic composition 120 may be incorporated into the core material 125. In some embodiments, the analgesic composition 120 is both incorporated into the core material 125 and coated thereon. The analgesic composition 120 comprises one or more analgesic compounds. The one or more analgesic compounds may comprise bupivacaine, lidocaine, prilocaine, benzocaine, dibucaine, pramoxine, phenol, cocaine, tetracaine, or any combination thereof. It is noted that, in embodiments where the analgesic composition 120 is coated onto the core material 125, it may be coated onto the core material 125 using the techniques described above. In one aspect, the core material 125 is coated using a solution comprising each of the one or more analgesic compounds in an amount of about 10 vol. % to about 30 vol. %. In a particular aspect, the solution may comprise each of the one or more analgesic compounds in an amount of about 20 vol. %. In embodiments where the analgesic composition 120 is incorporated into the core material 125, the analgesic composition 120 may be incorporated in a concentration of about 10% to about 30%. In a particular aspect, the analgesic composition 120 may be incorporated in a concentration of about 20%.

Further, according to some aspects, the analgesic nose plug 100 may further comprise one or more additional components. The additional components may be medicaments or may be compounds that provide another non-medical benefit. The one or more additional components may be incorporated into the core material 125 and/or into a coating provided on the core material 125. In embodiments where the analgesic composition 120 is coated onto the core material 125, the one or more additional components may be incorporated into the analgesic composition 120. In some aspects, the nose plug 100 may comprise one or more of the clotting factors, antibiotics, antimicrobial agents, antihistamines, decongestants, emollients, and antioxidants described above. The nose plug 100 may also comprise a corticosteroid such as hydrocortisone, mometasone, fluticasone, budesonide, betamethasone, and the like; an anti-dependency agent such as naloxone, naltrexone, methadone, buprenorphine, and the like; an odorant such as menthol, peppermint, salicylate, methyl salicylate, and the like; or any combination thereof.

Suitable packaging for the nose plugs disclosed herein include blister packages containing multiple numbers of the present nose plugs wherein each nose plug is sealed within a single blister. Such blister packages may contain one nose plug or two nose plugs or three nose plugs or four nose plugs or five nose plugs or six nose plugs or seven nose plugs or eight nose plugs or more. Such blister packages may then be sealed into a cardboard container, for example one blister package per container or two blister packages per container or three blister packages per container or four blister packages per container or more. Alternatively, multiples of the present nose plugs may be sealed in plastic bags that are then sealed in a cardboard container. Alternatively, multiples of the present nose plugs may be sealed into a plastic bottle or composite bottle or pressed fiber bottle. The multiples of the present nose plugs may be sealed in plastic bags before being sealed in the bottles. It is optional if so desired, to sterilize the sealed and packed nose plugs prior to distribution, for example by gamma ray irradiation.

Although the invention has been described in detail with particular reference to these embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference

1. A deformable analgesic nose plug, comprising:
a deformable core material comprising a multi-phasic core material wherein a first phase of the multi-phasic core material is characterized by a solid or a semi-solid structure at a temperature of 35° C. or less, and wherein a second phase of the multi-phasic core material is characterized by liquefaction at a temperature of 37° C. or greater; and
one or more analgesic compounds,
wherein the nose plug has a tapered distal end for insertion into a nostril and a proximal end whereby the nose plug is inserted into the nostril.

2. The deformable nose plug according to claim 1, wherein the one or more analgesic compounds are incorporated into the deformable core material and/or an outer coating provided on the deformable core material.

3. The deformable nose plug according to claim 1, wherein the one or more analgesic compounds comprise bupivacaine, lidocaine, prilocaine, benzocaine, dibucaine, pramoxine, phenol, cocaine, or tetracaine.

4. The deformable nose plug according to claim 1, further comprising one or more additional components that are an antihistamine, a decongestant, an antibiotic, an antimicrobial, a corticosteroid, an emollient, an anti-dependency agent, a odorant, or an antioxidant.

5. The deformable nose plug according to claim 4, wherein the one or more additional components are incorporated into an outer coating provided on the deformable core material and/or the deformable core material.

6. The deformable nose plug according to claim 1, wherein the deformable core material comprises a porous resiliently compressible substrate.

7. The deformable nose plug according to claim 6, wherein the porous resiliently compressible substrate is one of polymeric sponges, naturally occurring sponges, fluffy cellulosic materials, cotton, rolled gauze, and mixtures thereof.

8. The deformable nose plug according to claim 1, additionally having an applicator embedded into the deformable core material, said applicator extending outward from the proximal end of the nose plug.
